(19) United States
(12) Patent Application Publication
(10) Pub. No.: US 2024/0216228 A1
(43) Pub. Date: Jul. 4, 2024

(54) CALCIUM AND FLUORINE IONS RELEASING DENTAL COMPOSITION

(71) Applicant: 3M INNOVATIVE PROPERTIES COMPANY, St. Paul, MN (US)

(72) Inventors: Kai U. Claussen, Duesseldorf (DE); Johannes M. Leykauff, Weilheim (DE); Gioacchino Raia, Tuerkenfeld (DE); Reinhold Hecht, Schondorf am Ammersee (DE); Randilynn B. Christensen, Pine Springs, MN (US)

(21) Appl. No.: 18/556,736

(22) PCT Filed: Mar. 20, 2022
(86) PCT No.: PCT/IB22/52959

§ 371 Date: Oct. 23, 2023

(30) Foreign Application Priority Data
Apr. 29, 2021 (EP) 21171077.7

(51) Int. Cl. A61K 6/30; A61K 6/802; A61K 6/889

(52) U.S. Cl. CPC A61K 6/30; A61K 6/802; A61K 6/889

(57) ABSTRACT

The invention relates to a dental composition comprising polymerizable components, an initiator suitable for curing the polymerizable components, a Ca-ions releasing component having a Ca-ions releasing capability of 400 to 700 mg/l (ppm) if 2.5 g Ca-ions releasing component is steered in 50 ml de-ionized water having a pH of 2 for 24 hours; a F-ions releasing component having a F-ions releasing capability of 1,500 to 3,500 mg/l (ppm) if 2.5 g F-ions releasing component is steered in 50 ml de-ionized water having a pH of 7 for 24 hours. The invention also relates to a kit of parts comprising such a dental composition according and the following items alone or in combination: dental adhesive, dental milling block, prefabricated dental crown. The dental composition is suitable for use in a process of remineralizing a defect tooth in the mouth of a mammal, the process comprising the step of fixing a dental restoration to the surface of the defect tooth with the dental composition.

### FIELD OF THE INVENTION

The invention relates to a dental composition, in particular a resin-modified glass ionomer cement (RM-GIC) having calcium ions and fluoride ions releasing properties.

The dental composition can be used for cementing a dental restoration to a tooth structure and remineralizing the tooth structure to which the dental restoration is cemented.

### BACKGROUND

Dental caries refers to tooth decay in teeth which is typically caused by bacteria located in biofilms in the mouth of a patient. These bacteria produce inter alia lactic acid, which interacts with the hard dental tissue.

Hard dental tissue, in particular dental enamel contains a high amount of the phosphate mineral apatite having the general formula Ca₅(PO₄)₃(F,Cl,OH), wherein hydroxylapatite is the main component of the dental enamel substance. Compared to hydroxylapatite, fluoroapatite is said to be more resistant to acids.

Thus, to make hard dental tissue more resistant to acids it has been suggested to treat the hard dental tissue with compositions containing fluoride releasing components.

In this respect various mouth washes and toothpastes are available for daily practice.

Besides these products, there are also dental restorative compositions containing fluoride releasing components or phosphate releasing components. These dental compositions are typically used by the practitioner when treating a defect dental tooth.

E.g., US 2007/183984 A1 (Wrigley) describes an oral composition comprising a calcium phosphate salt and a combination of acids having differing solubilities in the oral cavity, for tooth mineralization or remineralization. The presence of a combination of acids in the oral composition is said to maximize the release of calcium and phosphate ions from the oral composition over an extended period of time, in order to promote the precipitation of enamel-like crystals on the surfaces of the teeth, or in subsurface regions therein.

U.S. Pat. No. 5,824,720 (Gangnus et al.) relates to fluoride-releasing, polymerizable dental composite materials containing (a) one or more ethylenically unsaturated polymerizable monomers based on di- or multi-functional (meth)acrylates; (b) initiators and optionally activators; (c) usual fillers, and optionally pigments, thixotropic agents, plasticizers and other auxiliaries; and (d) one or more sufficiently water-soluble inorganic complex fluorides of the general formula AₙMFₘ wherein, A is a monovalent cation, M is a metal of the III-V main group or II-V sub-group, n is a whole number from 1 to 3 and m is whole number from 3 to 6.

WO2018/102484A1 (3M) describes a hardenable dental composition comprising (e.g. a first part comprising) an encapsulated material wherein the encapsulated material comprises a basic core material and an inorganic shell material comprising a metal oxide surrounding the core; and (e.g. a second part comprising) water or an acidic component suitable for use in biological carrier materials.

WO2014/148002A1 (Kuraray) describes a resin-reinforced glass ionomer cement exhibiting release of calcium and fluoride ions containing fluoroaluminosilicate glass particles, acidic calcium phosphate and basic calcium phosphate particles or calcium compound particles excluding phosphorous, polyalkenoic acid, a non-acidic polymerizable monomer, water and a polymerization initiator. U.S. Pat. No. 4,746,686 (Waller) relates to a visible light activated cavity liner providing a source of leachable calcium and fluoride, the composition comprising a photopolymerizable matrix material, a photo initiator, a reducing agent, a synthetic hydroxyapatite filler and a powdered glass ionomer filler.

### SUMMARY OF INVENTION

However, there is still a need for a curable dental composition, in particular a dental cement addressing the task of remineralizing defect tooth structure in a more effective or different way.

In particular, there is a desire for a dental composition being able to release not only fluoride ions but also calcium ions.

Providing such a composition is not trivial, as once released, calcium and fluoride ions typically form water-insoluble calcium fluoride.

It would also be desirable if the dental composition has good adhesion properties.

Further, if possible, the dental composition should meet aesthetic expectations of the practitioner.

Ideally, the dental composition should also be sufficiently storage stable.

One or more of the above objects can be achieved by the invention described in the present text.

In one embodiment the present invention features a dental composition comprising polymerizable components, initiator suitable for curing the polymerizable components, a Ca-ions releasing component having a Ca-ions releasing capability of 400 to 700 mg/l (ppm) if 2.5 g Ca-ions releasing component is steered in 50 ml de-ionized water having a pH of 2 for 24 hours, a F-ions releasing component having a F-ions releasing capability of 1,500 to 3,500 mg/l (ppm) if 2.5 g F-ions releasing component is steered in 50 ml de-ionized water having a pH of 7 for 24 hours.

The dental composition is typically provided as a kit of parts comprising an acidic part and a non-acidic part.

The invention is also directed to a kit of parts comprising the dental composition described in the present text and the following items alone or in combination: dental adhesive, dental milling block, prefabricated dental crown.

The invention is also related to a dental composition for use in a process of remineralizing a defect tooth in the mouth of a mammal, the process comprising the step of fixing a dental restoration to the surface of the defect tooth with the dental composition described in the present text.

Unless defined differently, for this description the following terms shall have the given meaning:

The term “compound” or “component” is a chemical substance which has a certain molecular identity or is made of a mixture of such substances, e.g., polymeric substances.

A “hardenable or curable or polymerizable component” is any component which can be cured or solidified in the presence of a photo-initiator by radiation-induced polymerization. A hardenable component may contain only one, two, three or more polymerizable groups. Typical examples of polymerizable groups include unsaturated carbon groups, such as a vinyl group being present i.a. in a (methyl)acrylate group.

As used herein, “(meth)acryl” is a shorthand term referring to “acryl” and/or “methacryl”. For example, a “(meth) acryloxy” group is a shorthand term referring to either an acryloxy group (i.e., CH₂═CH—C(O)—O—) and/or a methacryloxy group (i.e., CH₂═C(CH₃)—C(O)—O—).

As used herein, “hardening” or “curing” a composition are used interchangeably and refer to polymerization and/or crosslinking reactions including, for example, photo-polymerization reactions and chemical-polymerization techniques (e. g., ionic reactions or chemical reactions forming radicals effective to polymerize ethylenically unsaturated compounds) involving one or more materials included in the composition.

An “initiator” is a substance being able to start or initiate the curing process of polymerizable components or monomers, e.g. redox/auto-cure chemical reaction or by a radiation induced reaction or by a heat induced reaction.

A “redox initiator system” is defined as the combination of reducing agent(s) and oxidizing agent(s) being located on the application part of the application device. If present, transition metal component(s) are also regarded as components of the redox initiator system.

“Dental article” means an article which is to be used in the dental field, especially as or for producing a dental restoration. A dental article has typically two different surface portions, an outer surface and an inner surface. The outer surface is the surface which is typically not in permanent contact with the surface of a tooth. In contrast thereto, the inner surface is the surface which is used for attaching or fixing the dental article to a tooth. If the dental article has the shape of a dental crown, the inner surface has typically a concave shape, whereas the outer surface has typically a convex shape. A dental article should not contain components which are detrimental to the patient's health and thus free of hazardous and toxic components being able to migrate out of the dental or orthodontic article.

“Dental restoration” means dental articles which are used for restoring a tooth to be treated. Examples of dental restorations include crowns, bridges, inlays, onlays, veneers, facings, copings, crown and bridged framework, and parts thereof.

A “particle” means a substance being a solid having a shape which can be geometrically determined. The shape can be regular or irregular. Particles can typically be analysed with respect to e.g. particle size and particle size distribution.

The particle size (d50) of a powder can be obtained from the cumulative curve of the grain size distribution. Respective measurements can be done using commercially available granulometers (e.g. Malvern Mastersizer 2000). “D” represents the diameter of powder particles and “50” refers to the volume percentage of the particles. Sometimes, the 50% is also expressed as “0.5”. For example, “(d50)=1 μm” means that 50% of the particles have a size of 1 μm or less.

“Paste” shall mean a soft, viscous mass of solids dispersed in a liquid.

“Viscous” means a viscosity above 50 Pa*s (at 23° C.).

A “liquid” means any solvent or liquid being able to at least partially disperse or dissolve a component at ambient conditions (e.g. 23° C.). A liquid typically has a viscosity below 10 or below 8 or below 6 Pa*s.

“Glass ionomer cement” (GIC) means a cement which cures or hardens by the reaction of an acid-reactive glass and a polyacid in the presence of water.

“Resin-modified glass ionomer cement” or “RM-GIC” shall mean a GIC containing in addition polymerizable component(s), an initiator system and typically a diluting agent such as 2-hydroxyl-ethyl-methacrylate (HEMA).

“Acid-reactive filler” shall mean a filler that chemically reacts in the presence of a (poly)acid leading to a hardening reaction.

“Non acid-reactive filler”: shall mean a filler, which does not show a chemical hardening reaction within about 30 min, if mixed with a (poly)acid at ambient conditions (e.g. 23° C.).

To distinguish an acid-reactive filler from a non acid-reactive filler the following test can or is to be conducted: A composition is prepared by mixing Part A with Part B in a mass ratio of 3 to 1, wherein: Part A contains: filler to be analysed: 100 wt. %; Part B contains: poly (acrylic acid co maleic acid) (Mw: about 20,000+/−3,000): 43.6 wt. %, water: 47.2 wt. %, tartaric acid: 9.1 wt. %, benzoic acid: 0.1 wt. %.

“Polyacid” or “polyalkenoic acid” shall mean a polymer having a plurality of acidic repeating units (e.g. more than 10 or more than 20 or more than 50). That is, the acidic repeating units are attached to or pending from the backbone of the polymer.

A “storage stable composition” is a composition which can be stored for an adequate period of time (e.g. at least 12 months under ambient conditions or 3 months under accelerated aging conditions) without showing significant performance issues (e.g. reduced flexural or compressive strength), and/or which does not harden over time and/or which does not separate over time.

As used herein, a “dental surface” refers to tooth structures (e. g., enamel, dentin, and cementum) and bone.

A “self-etching” composition refers to a composition which bonds to a dental surface without pre-treating the dental surface with an etchant. Preferably, a self-etching composition can also function as a self-adhesive primer wherein no separate etchant or primer is used.

A “self-adhesive” composition refers to a composition that is capable of bonding to a dental surface without pre-treating the dental surface with a primer or bonding agent. Preferably, a self-adhesive composition is also a self-etching composition wherein no separate etchant is used.

A “self-curing composition” means a composition which cures by a redox-reaction without application of radiation.

An “untreated” dental surface refers to a tooth or bone surface that has not been treated with an etchant, primer, conditioner, or bonding agent prior to application of a self-etching adhesive or a self-adhesive composition.

An “unetched” dental surface refers to a tooth or bone surface that has not been treated with an etchant prior to application of a self-etching adhesive or a self-adhesive composition.

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar (at maritime level).

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

A composition is “essentially or substantially free of” a certain component, if the composition does not contain said component as an essential feature. Thus, said component is not willfully added to the composition either as such or in combination with other components or ingredient of other components. A composition being essentially free of a certain component usually does not contain that component at all. However, sometimes the presence of a small amount of the said component is not avoidable e.g. due to impurities contained in the raw materials used.

### DETAILED DESCRIPTION

It has been found that the composition described in the text has a couple of advantageous properties.

The dental composition is capable of releasing calcium and fluoride ions in combination.

The release of fluoride ions from dental materials and toothpastes is perceived beneficial because the presence of fluoride is assumed to correlate with improved oral health. It is assumed that the hardness of the tooth surface structure can be improved if fluoride ions are applied.

This can typically be achieved by either using fluoride ions releasing reactive glasses which are often present in RM-GICs or by adding fluoride ions containing compounds such as sodium fluoride to dental compositions.

It is also assumed that the application of calcium ions to a tooth surface may also contribute to the remineralization of the tooth structure.

This can typically be achieved by adding water-soluble calcium salts. However, not all kinds of Ca-ions releasing components or F-ions releasing components are suitable.

It was found that it is challenging to provide a significant release of calcium in the presence of fluoride, presumably due to the in-situ formation of insoluble calcium fluoride. In fact, RM-GIC formulations containing sodium fluoride were found to release less calcium than formulations without sodium fluoride.

Without wishing to be bound to a certain theory it is assumed that the respective components should have a kind of delayed ion releasing capability so that the solubility product of calcium fluoride is not exceeded during the preparation of the dental composition and its application to a prepared tooth structure.

Thus, the Ca-ions and the F-ions are still available for being incorporated in the dental hard tissue and thus can contribute to the remineralization of the hard dental tissue over time.

Due to this property the dental composition can also be regarded as a so-called bioactive material.

Further, depending on the nature of the initiator systems used, the dental composition can also easily be cured, either by applying radiation, or is self-curing or can be cured by using different curing reactions in combination.

The dental composition described in the present text is also sufficiently storage stable and has sufficient adhesion properties (e.g. shear bond strength) if formulated as self-adhesive composition.

By choosing the appropriate components, also the aesthetic aspects of the dental composition can be addressed.

If desired, the dental composition described in the present text can typically be characterized by the following properties alone or in combination:
a) viscosity: 10 to 5,000 Pa*s at 28° C., or 100 to 2,000 Pa*s at 28° C., measured in oscillation at a frequency of 1.25 Hz and deflection of 1.75% 60 s after starting to mix the composition;
b) pH value: 1 to 6; e.g. if determined with a wet pH-sensitive indicator paper or stick;
c) shear bond strength to dentin: 1 to 10 MPa;
d) setting time: within 10 min measured at 28° C.

The combination of the following properties is sometimes preferred: a) and b); a) and c); b) and c).

The dental composition comprises polymerizable components. A polymerizable component comprises a component with at least one or two polymerizable moieties such as a (meth)acrylate moiety. The crosslinking or polymerization of the polymerizable component can be initiated by using a redox-initiator system and/or by using a photo initiator system.

In some embodiments the polymerizable components contain acidic groups or moieties.

The polymerizable components with acid moiety can typically be represented by the following formula

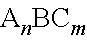

with A being an ethylenically unsaturated group, such as a (meth)acryl moiety, B being a spacer group, such as (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), (iii) organic group having 4 to 20 carbon atoms bonded to one another by one or more ether, thioether, ester, thioester, thiocarbonyl, amide, urethane, carbonyl and/or sulfonyl linkages, and C being an acidic group, or precursor of an acidic group such as acid anhydride, m, n being independently selected from 1, 2, 3, 4, 5 or 6, wherein the acidic group comprises one or more carboxylic acid residues, such as —COOH or —CO—O—CO—, phosphoric acid residues, such as —O—P(O)(OH)OH, phosphonic acid residues, such as C—P(O)(OH)(OH), sulfonic acid residues, such as —SO₃H or sulfinic acid residues such as —SO₂H.

Examples of polymerizable components with acid moiety include, but are not limited to glycerol phosphate mono(meth)acrylate, glycerol phosphate di(meth)acrylate, hydroxyethyl (meth)acrylate (e.g., HEMA) phosphate, bis((meth)acryloxyethyl) phosphate, (meth)acryloxypropyl phosphate, bis((meth)-acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylate. Derivatives of these hardenable components bearing an acid moiety that can readily react e.g. with water to form the specific examples mentioned above, like acid halides or anhydrides are also contemplated.

Polymerizable components with acidic groups are typically present in the following amounts: at least 0.5 or 1 or 2 wt. %; utmost 15 or 10 or 8 wt. %; range: 0.5 to 15 or 2 to 10 or 3 to 8 wt. %; wt. % with respect of the weight of the dental composition.

In some embodiments the polymerizable components does not contain acidic groups.

The polymerizable component without an acidic moiety is typically a free-radically polymerizable material, including ethylenically unsaturated monomer, monomers or oligomers or polymers.

Suitable polymerizable component(s) without acidic moiety(s) can be characterized by the following formula:

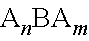

with A being an ethylenically unsaturated group, such as a (meth)acryl moiety,
B being selected from (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), or (iii) organic group having 4 to 20 carbon atoms bonded to one another by one or more ether, thioether, ester, thioester, thiocarbonyl, amide, urethane, carbonyl and/or sulfonyl linkages,
m, n being independently selected from 0, 1, 2, 3, 4, 5 or 6 with the proviso that n+m is greater 0, that is that at least one A group is present.

Such polymerizable materials include mono-, di- or poly-acrylates and methacrylates such as methyl acrylate, methyl methacrylate, ethyl (meth)acrylate, isopropyl (meth)acrylate, n-hexyl (meth)acrylate, stearyl (meth)acrylate, allyl (meth)acrylate, glycerol di(meth)acrylate, the diurethane dimethacrylate called UDMA (mixture of isomers, e.g. Rohm Plex 6661-0) being the reaction product of 2-hydroxyethyl methacrylate (HEMA) and 2,2,4-trimethylhexamethylene diisocyanate (TMDI), glycerol tri(meth)acrylate, ethyleneglycol di(meth)acrylate, diethyleneglycol di(meth)acrylate, triethyleneglycol di(meth)acrylate, 1,3-propanediol diacrylate, 1,3-propanediol dimethacrylate, trimethylolpropane tri(meth)acrylate, 1,2,4-butanetriol tri(meth)acrylate, 1,4-cyclohexanediol di(meth)acrylate, pentaerythritol tri(meth)acrylate, pentaerythritol tetraacrylate, pentaerythritol tetramethacrylate, sorbitol hexa(meth)acrylate, bis[1-(2-(meth)acryloxy)]-p-ethoxyphenyldimethylmethane, bis[1-(3-methacryloxy-2-hydroxy)]-p-propoxyphenyldimethylmethane (BisGMA), bis[1-(3-acryloxy-2-hydroxy)]-p-propoxy-phenyldimethylmethane and trishydroxyethyl-isocyanurate trimethacrylate; the bis-acrylates and bis-methacrylates of polyethylene glycols of molecular weight 200-500, copolymerizable mixtures of acrylated monomers (see e.g. U.S. Pat. No. 4,652,274), and acrylated oligomers (see e.g. U.S. Pat. No. 4,642,126); and vinyl compounds such as styrene, diallyl phthalate, divinyl succinate, divinyl adipate and divinylphthalate; polyfunctional (meth)acrylates comprising urethane, urea or amide groups. Mixtures of two or more of these free radically polymerizable materials can be used if desired.

Further polymerizable components which may be present include di(meth)acrylates of ethoxylated bis-phenol A, for example 2,2′-bis(4-(meth)acryloxytetraethoxyphenyl)propanes, urethane (meth)acrylates and (meth)acrylamides. The monomers used can furthermore be esters of [alpha]-cyanoacrylic acid, crotonic acid, cinnamic acid and sorbic acid.

It is also possible to use the methacrylic esters mentioned in EP 0 235 826, such as bis[3[4]-methacryl-oxymethyl-8(9)-tricyclo[5.2.1.0^{2,6}]decylmethyl triglycolate. Suitable are also 2,2-bis-4(3-methacryloxy-2-hydroxypropoxy)phenylpropane (Bis-GMA), 2,2-bis-4(3-methacryloxypropoxy)phenyl-propane, 7,7,9-trimethyl-4,13-dioxo-3,14-dioxa-5,12-diazahexadecane-1,16-dioxy dimethacrylate (UDMA), urethane (meth)acrylates and di(meth)acrylates of bishydroxymethyltricyclo-(5.2.1.0²,6)decane.

These ethylenically unsaturated monomers can be employed in the dental composition(s) either alone or in combination with the other ethylenically unsaturated monomers. In addition or besides those components, other hardenable components which can be added include oligomeric or polymeric compounds, such as polyester (meth)acrylates, polyether (meth)acrylates, polycarbonate (meth)acrylates and polyurethane (meth)acrylates. The molecular weight of these compounds is typically less than 20,000 g/mol, particularly less than 15,000 g/mol and in particular less than 10,000 g/mol.

Polymerizable components without acidic groups are typically present in the following amounts: at least 1 or 5 or 10 wt. %; utmost 50 or 45 or 40 wt. %; range: 1 to 50 or 5 to 45 or 10 to 40 wt. %; wt. % with respect of the weight of the dental composition.

The dental composition comprises an initiator suitable for curing the polymerizable components. Different kinds of initiator systems can be used.

The dental composition may comprise a photo-initiator or photo-initiator system and/or a redox-initiator system.

According to certain embodiments, the dental composition comprises a photo-initiator.

As photo-initiator(s), those which can polymerize the polymerizable monomer(s) by the action of visible light having a wavelength of in the range of 350 nm to 500 nm are preferred.

Suitable photo-initiator(s) often contain an alpha di-keto moiety, an anthraquinone moiety, a thioxanthone moiety or benzoin moiety.

Examples of photo-initiator(s) include camphor quinone, 1-phenyl propane-1,2-dione, benzil, diacetyl, benzyl dimethyl ketal, benzyl diethyl ketal, benzyl di(2-methoxyethyl) ketal, 4,4,′-di-methylbenzyl dimethyl ketal, anthraquinone, 1-chloroanthraquinone, 2-chloroanthraquinone, 1,2-benz-anthraquinone, 1-hydroxyanthraquinone, 1-methylanthraquinone, 2-ethylanthraquinone, 1-bromoanthraquinone, thioxanthone, 2-isopropyl thioxanthone, 2-nitrothioxanthone, 2-methyl thioxanthone, 2,4-dimethyl thioxanthone, 2,4-diethyl thioxanthone, 2,4-diisopropyl thioxanthone, 2-chloro-7-trifluoromethyl thioxanthone, thioxanthone-10,10-dioxide, thioxanthone-10-oxide, benzoin methyl ether, benzoin ethyl ether, isopropyl ether, benzoin isobutyl ether, benzophenone, bis(4-dimethyl-aminophenyl)ketone, 4,4,′-bisdiethylaminobenzophenone.

Using acylphosphine oxides was found to be useful, as well.

Suitable acylphosphine oxides can be characterized by the following formula

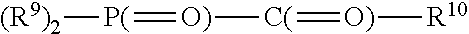

wherein each R⁹ individually can be a hydrocarbyl group such as alkyl, cycloalkyl, aryl, and aralkyl, any of which can be substituted with a halo-, alkyl- or alkoxy-group, or the two R⁹ groups can be joined to form a ring along with the phosphorous atom, and wherein R¹⁰ is a hydrocarbyl group, an S-, O-, or N-containing five- or six-membered heterocyclic group, or a —Z—C(═O)—P(═O)— (R⁹)₂ group, wherein Z represents a divalent hydrocarbyl group such as alkylene or phenylene having 2 to 6 carbon atoms.

Suitable systems are also described e.g. in U.S. Pat. No. 4,737,593 (Ellrich et al.), the content of which is herewith incorporated by reference.

Preferred acylphosphine oxides are those in which the R⁹ and R¹⁰ groups are phenyl or lower alkyl- or lower alkoxy-substituted phenyl. By “lower alkyl” and “lower alkoxy” is meant such groups having from 1 to 4 carbon atoms. In particular, 2,4,6-trimethylbenzoyl diphenyl phosphine oxide was found to be useful (Lucirin™ TPO, BASF).

More specific examples include: bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenyl-phosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)-phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenyl-phosphine oxide, bis-(2-methyl-1-naphthoyl)phenylphosphine oxide, bis-(2-methyl-1-naphthoyl)-4-biphenylylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenyl-phosphine oxide, bis-(2-methoxy-1-naphthoyl)-4-biphenylylphosphine oxide, bis-(2-methoxy-1-naphthoyl)-2-naphthylphosphine oxide and bis-(2-chloro-1-naphthoyl)-2,5-dimethylphenylphosphine oxide.

The acylphosphine oxide bis(2,4,6-trimethylbenzoyl)phenyl phosphine oxide (previously known as IRGACURE™ 819, Ciba Specialty Chemicals) is sometimes preferred.

A photo-initiator may be present in the following amounts: at least 0 or 0.005 or 0.01 wt. %; utmost 2 or 1.5 or 1 wt. %; range: 0 to 2 or 0.001 to 1.5 or 0.01 to 1 wt. %; wt. % with respect of the weight of the dental composition.

According to certain embodiments, the dental composition comprises a redox-initiator system.

A redox-initiator system typically comprises oxidizing agent(s) and reducing agent (s) and sometimes transition metal component(s).

Suitable oxidizing agents include organic peroxide and persulfate component(s) and mixtures thereof.

Organic peroxides which can be used include hydroperoxide(s), ketone peroxide(s), diacyl peroxide(s), dialkyl peroxide(s), peroxyketal(s), peroxyester(s) and peroxydicarbonate(s).

Di-peroxides, which can be used include di-peroxides comprising the moiety R₁—O—O—R₂—O—O—R₃, with R₁ and R₃ being independently selected from H, alkyl (e.g. C₁ to C₆), branched alkyl (e.g. C₁ to C₆), cycloalkyl (e.g. C₅ to C₁₀), alkylaryl (e.g. C₇ to C₁₂) or aryl (e.g. C₆ to C₁₀) and R₂ being selected from alkyl (e.g. (C₁ to C₆) or branched alkyl (e.g. C₁ to C₆).

Examples of ketone peroxides include methyl ethyl ketone peroxide, methyl isobutyl ketone peroxide, methyl cyclohexanone peroxide, and cyclohexanone peroxide.

Examples of peroxyesters include cumylperoxyneodecanoate, t-butyl peroxypivarate, t-butyl peroxyneodecanoate, 2,2,4-trimethylpentylperoxy-2-ethyl hexanoate, t-amylperoxy-2-ethyl hexanoate, t-butylperoxy-2-ethyl hexanoate, di-t-butylperoxy isophthalate, di-t-butylperoxy hexahydroterephthalate, t-butylperoxy-3,3,5-trimethylhexanoate, t-butylperoxy acetate, t-butylperoxy benzoate and t-butylperoxymaleic acid.

Examples of peroxidicarbonates include di-3-methoxy peroxidicarbonate, di-2-ethylhexyl per-oxydicarbonate, bis(4-t-butylcyclohexyl)peroxidicarbonate, diisopropyl-1-peroxydicarbonate, di-n-propyl peroxidicarbonate, di-2-ethoxyethyl-peroxidicarbonate, and diallyl peroxidicarbonate.

Examples of diacyl peroxides include acetyl peroxide, benzoyl peroxide, decanoyl peroxide, 3,3,5-trimethylhexanoyl peroxide, 2,4-dichlorobenzoyl peroxide and lauroylperoxide.

Examples of dialkyl peroxides include di-t-butyl peroxide, dicumylperoxide, t-butylcumyl peroxide, 2,5-dimethyl-2,5-di(t-butylperpoxy)hexane, 1,3-bis(t-butylperoxyisopropyl)benzene and 2,5-dimethyl-2,5-di(t-butylperoxy)-3-hexane.

Examples of peroxyketals include 1,1-bis(t-butylperoxy)-3,3,5-trimethylcyclohexane, 1,1-bis(t-butylperoxy)cyclohexane, 2,2-bis(t-butylperoxy)butane, 2,2-bis(t-butylperoxy)octane and 4,4-bis(t-butylperoxy)valeric acid-n-butylester.

According to one embodiment, the organic peroxide is a hydroperoxide, in particular a hydroperoxide comprising the structural moiety R—O—O—H with R being (e.g. C₁ to C₂₀) alkyl, (e.g. C₃ to C₂₀) branched alkyl, (e.g. C₆ to C₁₂) cycloalkyl, (e.g. C₇ to C₂₀) alkylaryl or (e.g. C₆ to C₁₂) aryl.

Examples of suitable organic hydroperoxides include t-butyl hydroperoxide, t-amyl hydroperoxide, p-diisopropylbenzene hydroperoxide, cumene hydroperoxide, pinane hydroperoxide, p-methane hydroperoxide and 1,1,3,3-tetramethylbutyl hydroperoxide.

Suitable peroxodisulfate components and/or peroxodiphosphate components and/or mixtures thereof, which can be used include organic and/or inorganic components.

Suitable examples include ammonium, sodium, and potassium peroxodisulfate components and/or peroxodiphosphate components. Sodium peroxodisulfate is sometimes preferred.

Suitable transition metal component(s) include organic and/or inorganic salt(s) selected from titanium, vanadium, chromium, manganese, iron, cobalt, nickel, copper and/or zinc, with copper and iron being sometimes preferred.

Useful salts include acetate(s), chloride(s), sulphate(s), benzoate(s), acetylacetonate(s), naphthenate(s), carboxylate(s), bis(1-phenylpentan-1,3-dione) complexes, salicylate(s), complexes with ethylenediaminetetraacetic acid of either of the transition metals and mixtures thereof.

According to one embodiment, the transition metal component is in an oxidation stage, which allows the component to be reduced. Useful oxidation stages include +2, +3, +4, +5, +6 and +7, as applicable.

Copper component(s) are sometimes preferred. The oxidation stage of copper in the copper component(s) is preferably +1 or +2.

Typical examples of copper component(s) which can be used include salts and complexes of copper including copper acetate, copper chloride, copper benzoate, copper acetylacetonate, copper naphthenate, copper carboxylates, copper bis(1-phenylpentan-1,3-dione) complex (copper procetonate), copper salicylate, complexes of copper with thiourea, ethylenediaminetetraacetic acid and/or mixtures thereof. The copper compounds can be used in hydrated form or free of water. Especially preferred is copper acetate.

The amount of transition metal component which can be used is not particularly limited. The transition metal salt should be used in an amount sufficient to achieve the intended purpose.

Reducing agents (s) include ascorbic acid component(s), tertiary amine component(s), sulfinate component(s), sulphite component(s), borane component(s), (thio)urea component(s), and (thio)barbituric acid component(s), saccharin and metal salts thereof.

Preferred are also thiourea components comprising a polymerizable moiety, in particular a (meth)acrylate moiety.

The polymerizable thiourea component comprises a thiourea moiety which is attached to a (meth)acrylate moiety through a spacer unit comprising an alkyl chain, preferably a C₃₋₁₁ alkyl chain.

The molecular weight of the polymerizable thiourea component is typically in a range of 200 to 400 g/mol. This molecular weight range seems to be a good compromise to allow a sufficient molecular mobility and reactivity within a curable composition to be cured.

The polymerizable thiourea component may be characterized by the following formula:

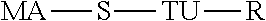

with
MA: (meth)acrylate,
S: C₃₋₁₁ linear alkyl, or C₃₋₈ linear alkyl or C₃₋₆ linear alkyl,
TU: thiourea,
R: C₁ to C₆ alkyl or cyclo alkyl (C₃_6, preferably C₅-6).

The polymerizable thiourea component does typically not comprise an allyl moiety (H₂C═CH—CH₂—) and a carbamate moiety (—O—CO—NH—).

Suitable polymerizable thiourea component typically show an average water-contact angle in the range of more than 100 or 15° determined over a time period of 0 to 12 s after placement of a water droplet on a surface coated with the polymerizable thiourea component.

Preferred polymerizable thiourea components include N-(5-methacryloxypentyl)-N′-ethyl thiourea, N-(5-methacryloxypentyl)-N′-cyclohexyl thiourea, N-(5-methacryloxyundecyl)-N′-ethyl thiourea and mixtures thereof.

If the oxidizing agents(s) are brought in contact with the reducing agents(s), a redox-reaction typically starts. Such a redox-reaction is suitable to initiate the curing of curable components resulting in the crosslinking of the curable components.

If a redox-initiator system comprising an oxidizing, a reducing component and optionally a transition metal component is present, it is typically present in the following amounts: at least 0.1 or 0.2 or 0.3 wt. %; utmost 5 or 4 or 3 wt. %; range: 0.1 to 5 or 0.2 to 4 or 0.3 to 3 wt. %; wt. % with respect of the weight of the dental composition.

The dental composition comprises a calcium ions releasing component.

The Ca-ions releasing component has a Ca-ions releasing capability of 400 to 700 mg/L if 2.5 g of the compound are stirred in 50 mL deionized water for 24 h at pH=2.

The Ca-ions releasing component is typically present in the following amounts: at least 0.5 or 1 or 2 wt. %; utmost 30 or 25 or 20 wt. %; range: 0.5 to 30 or 1 to 25 or 2 to 20 wt. %; wt. % with respect of the weight of the dental composition.

According to some embodiments, the Ca-ions releasing component comprises a basic core material that releases Ca-ions, and an inorganic shell material comprising a metal oxide surrounding the basic core material.

A basic core material is a material which provides a pH above 7 when dissolved in deionized water.

Examples of basic core materials include oxides and hydroxides of alkali and alkaline earth metals, as well as strongly basic salts, such as alkali phosphates.

Specific examples of basic core materials include oxides and hydroxides of Na, K, Ca, Sr, and Ba; silicates of Na, K, Ca, Sr, and Ba; and aluminates of Na, K, Ca, Sr, and Ba. Basic silicates and glasses typically comprise at least 1, 2, or 3 moles of basic core compound (e.g. CaO) per mole of silica on a cation molar basis. Likewise, basic aluminate typically comprises at least 1, 2, or 3 moles of basic core compound (e.g. CaO) per mole of alumina on a cation molar basis.

In some embodiments, the core comprises and is prepared from CaO, having a pKa of 11.6. The amount of CaO is typically at least 5, 10, 15, 20, or 25 wt. % and can range up to 75 wt. % or greater.

Specific examples of basic core materials comprising CaO include Portland cements (reported to contain 60-70% wt. % CaO); tricalcium silicate (containing about 75 wt. % CaO); and bioactive glass, as can be obtained from 3M Advanced Material Division (containing about 25 wt. % of CaO, and about 25 wt. % of Na₂O).

Conventional natural (e.g. Portland) and synthetic cements typically comprises a major amount of calcium silicate (e.g. 3CaO—SiO₂, 2CaO—SiO₂) alone or in combination with one or more calcium aluminates (e.g. 3CaO-Al₂O₃, 4CaO—Al₂O₃—Fe₂O3) and is curable or self-setting when mixed with water. Portland cement typically contains about 61%-69% CaO, about 18%-24% SiO₂, about 2%-6% Al₂O₃, about 1%-6% Fe₂O₃, about 0.5%-5% MgO.

The Ca-ions releasing component can be produced by providing basic core particles and encapsulating the basic core particles with a (e.g. continuous, non-particulate) inorganic coating by means of least one of a vapor deposition technique. Vapor deposition technique include chemical vapor deposition (CVD) such as atmospheric pressure chemical vapor deposition (APCVD), hydrolysis CVD, and plasma CVD).

Advantages of vapor deposition techniques for providing the coatings include that the coating is built up from molecular size species without interference from a solvent or liquid media. Some coating methods (e.g., ALD and CVD) tend to provide coatings composed of conformal layers on irregular materials (e.g., powder or porous particulate).

ALD and CVD are coating processes involving chemical reactions, where the chemical reactants used are referred to as chemical precursors. That is, they are precursors to the coating material (i.e., coating precursors) to be formed (e.g., a metal oxide coating). In some embodiments, a single coating precursor is used, while in other embodiments, at least two coating precursors are used. At least one coating precursor comprises at least one metal cation needed for the coating (e.g., a metal oxide coating).

An effective coating method for making encapsulated materials described herein is atmospheric pressure CVD (APCVD). APCVD can be carried out in simple equipment such as glassware. In some embodiments, hydrolysis reactions are used to form (e.g. continuous) metal oxide coatings at temperatures ranging from room temperature (ranging from about 22° C.) up to about 180° C.

Exemplary precursors for ALD and CVD processes include coating precursors (e.g., metal oxide precursors) comprising at least one metal cation such as metal alkyls (e.g., trimethyl or triethyl aluminium, diethyl zinc), volatile metal chlorides (titanium tetrachloride, silicon tetrachloride, aluminium trichloride), silane, metal alkoxides (titanium isopropoxide, aluminium isopropoxide, silicon ethoxide), compounds with mixed alkyl, halide, hydride, alkoxy, and other groups, and other volatile metallorganic compounds. Exemplary co-reactants to the coating precursor comprising at least one metal cation (e.g., a metal oxide precursor comprising at least one metal cation) include water, oxygen, ozone, ammonia, and alkyl amines. In addition to metal oxides, other inorganic, nonmetallic coating materials are deposited using chemical reactions between a coating precursor and a co-reactant to the coating precursor (e.g., a metal nitride coating deposited using a metal nitride precursor comprising at least one metal cation and a co-reactant to the metal nitride precursor).

Exemplary (e.g. continuous) coatings comprise, for example, nonmetallic, inorganic materials such as metal (e.g., Al, Si, Ti, Zr, Mg, and Zn) oxides. In some embodiments, the shell material comprises at least 50, 60, 70, 80, 90 or 100 wt.-% of a single metal oxide or combination thereof. Exemplary metal oxides may include forms such as hydroxides, and hydrous oxides, as well as forms with mixed anions (e.g., oxide plus halides, hydroxyls, small amounts of alkyls or carboxylates, etc.). The shell material is predominantly inorganic having a carbon content no greater than 20, 10, 5, or 1 wt. %. Further, the encapsulated basic material may also have a carbon content no greater than 20, 10, 5, or 1 wt. %. The shell material may further comprise metal nitrides, metal sulfides, metal oxysulfides, and metal oxynitrides. The coatings can be amorphous, crystalline, or mixed, single or multiphase, and can contain one or more cations and one or more anions. In some embodiments, the coating is amorphous alumina with or without some hydroxyls or bound water.

In some embodiments, the shell has an average thickness of at least 5, 10, 15, 20, or 25 nm. The thickness of the shell may range up to 250, 500, 750, or 1000 nm (1 micrometer). In some embodiments, such as in the case of encapsulated dental filler, the thickness of the shell typically ranges up to 100, 150, or 200 nm.

On a wt. % basis the shell material is typically at least 0.1, 0.2, 0.3, 0.4, or 0.5 wt. % of the total encapsulated material. The amount of shell material on a wt. % basis can range up to 15 or 20 wt. % of the total encapsulated material, but is more typically no greater than 10, 9, 8, 7, 6, or 5 wt. %.

A particularly useful Ca-ions releasing component comprises a basic core material comprising a CaO component, in particular calcium silicates such as 3CaO—SiO₂ and 2CaO—SiO₂, the surface of which is covered with an inorganic shell material comprising Al₂O₃ or SiO₂.

Suitable Ca-ion releasing components are also described in WO 2018/102484 A1 (3M). The content of this reference is herewith incorporated by reference.

The dental composition comprises a fluoride ions releasing component.

The F-ions releasing component of the present text has a F-ions releasing capability of 1,500 to 3,500 mg/l (ppm) if 2.5 g F-ions releasing component is steered in 50 ml de-ionized water for 24 hours.

The F-ions releasing component can typically be characterized by the following features alone or in combination: molecular weight: 110 to 300 g/mol; water solubility: 3 to 20 g/l water at 23° C.

If the molecular weight of the F-ions releasing component is too low, the water solubility is often too high.

The F-ions releasing component is typically present in the following amounts: at least 0.1 or 0.5 or 1 wt. %; utmost 20 or 15 or 10 wt. %; range: 0.1 to 20 or 0.5 to 15 or 1 to 10 wt. %; wt. % with respect of the weight of the dental composition.

Suitable F-ions releasing components which can be used include those having the formula

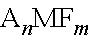

with
   A being an alkali metal ion or NR, with R being C₁ to C₁₈ alkyl, phenyl or substituted phenyl,
   M being selected from Ti, Zr, Al, Zn, P,
   n being 1 to 3,
   m being 3 to
F-ions releasing components which were found to be particular useful include KZnF₃, K₂TiF₆, and mixtures thereof.

These F-ions releasing components were found to be suitable to also address aesthetic expectations for a dental composition as they do not negatively affect the opacity of the cured dental composition.

The Ca-ions releasing component is typically contained in the dental composition in a higher amount (with respect to weight) compared to the amount of the F-ions releasing component.

The ratio of Ca-ions releasing component contained in the dental composition to F-ions releasing component being in a range of 1:0.9 to 1:0.1 or 1:0.7 to 1:0.3 with respect to weight.

The Ca-ions releasing component and the F-ions releasing component are typically present in the dental composition in amounts sufficient to enable a release of F-ions in an amount of at least 10 ppm per 1 g of cured composition stored in de-ionized water over 30 days at 36° C., and Ca-ions in an amount of at least 2 ppm per 1 g of cured composition stored in de-ionized water over 30 days at 36° C.

In certain embodiments the Ca-ions releasing component and the F-ions releasing component are present in the dental composition in amounts sufficient to enable a release of F-ions in an amount in the range of 10 ppm to 25 ppm per 1 g of cured composition stored in de-ionized water over 30 days at 36° C., and Ca-ions in an amount in the range of 2 ppm to 10 ppm per 1 g of cured composition stored in de-ionized water over 30 days at 36° C.

The above-mentioned amounts or ranges are considered sufficient for achieving the desired remineralization effect on the hard dental tissue.

The dental composition may also comprise one or more fillers. Suitable fillers include acid-reactive fillers, in particular acid-reactive glasses, and non acid-reactive fillers.

The acid-reactive glass is able to undergo a glass-ionomer cement reaction.

According to one embodiment, the acid-reactive glass can be characterized by the following features alone or in combination:
a) Mean particle size: 1 to 25 μm;
b) (d10/μm): 0.5 μm to 3 μm; (d50/μm): 2 μm to 7 μm; (d90/μm): 6 μm to 30 μm;
c) pH value of a dispersion of 1 g filler stirred in 10 ml de-ionized water for 5 minutes: between 5 and 10.

The combination of features a) and b) or a) and c) can be preferred.

If the mean particle size of the acid-reactive glass is above the range outlined above, the consistency of the composition obtained when mixing the compositions contained in the parts of the kit of parts described in the present text might not be adequate and the desired mechanical properties might be negatively affected.

If the mean particle size of the acid-reactive glass is below the range outlined above, the setting time might be too fast.

Suitable acid-reactive glasses include aluminosilicate glasses and fluoro aluminosilicate glasses (FAS glass).

The acid-reactive glass can be made from a melt containing fluoride, silica, alumina, and other glass-forming ingredients using techniques familiar to those skilled in the FAS glassmaking art.

The FAS glass typically is in the form of particles that are sufficiently finely divided so that they can conveniently be mixed with the other cement components and will perform well when the resulting mixture is used in the mouth.

Suitable FAS glasses are familiar to those skilled in the art and are available from a wide variety of commercial sources, and many are found in currently available glass ionomer cements such as those commercially available under the trade designations Ketac™-Molar or Ketac™-Fil Plus (3M Oral Care), and FUJI™ IX (GC).

Useful acid-reactive glasses can also be characterized by the Si/Al ratio. Fillers having a Si/Al ratio (by wt. %) of below 1.5 or 1.4 or 1.3 were found to be particularly useful.

Suitable acid-reactive glasses are also commercially available from e.g. Schott AG (Germany) or Specialty Glass (US).

If the amount of the acid-reactive glass is too high, mixing of the pastes of the kit of parts described in the present text might become more difficult. Furthermore, obtaining an adequate consistency and acceptable mechanical properties of the resulting composition might become difficult, as well.

If the amount of the acid-reactive glass is too low, formulating a suitable paste might become more difficult. Furthermore, the mechanical properties might become inferior.

The acid-reactive glass is typically present in the following amounts: at least 5 or 8 or 10 wt. %; utmost 70 or 60 or 50 wt. %; range: 5 to 70 or 8 to 60 or 10 to 50 wt. %; wt. % with respect of the weight of the dental composition.

According to one embodiment, the non acid-reactive filler can be characterized by the following features alone or in combination:
a) Mean particle size: 10 nm to 500 nm; or from 10 to 200 nm
b) Containing no particles larger than 2 μm;
c) pH value of a dispersion of 1 g filler stirred in 10 ml de-ionized water for 5 minutes: between 4 and 9.

The combination of features a) and b) or b) and c) is sometimes preferred.

If the mean particle size of the non acid-reactive filler is above the range outlined above, the consistency of the obtained paste might not be adequate and in addition it might become difficult to obtain the desired mechanical properties.

If the mean particle size of the non acid-reactive filler is below the range outlined above, the desired consistency of the obtained paste might not be adequate.

Examples of suitable non acid-reactive fillers are naturally occurring or synthetic materials including, but not limited to: kaolin; silica particles (e.g., submicron pyrogenic silicas such as those available under the trade designations “AEROSIL™”, including “OX 50,” “130,” “150” and “200”, silicas from Evonic, and HDK™, including “H15”, “H20”, “H2000” from Wacker, and CAB-O-SIL M5 silica from Cabot Corp.), alumina, titania and zirconia particles.

Mixtures of these non-acid-reactive fillers are also contemplated.

Sometimes, the non acid-reactive filler is provided as a dispersion or sol of particles in a liquid (e.g. water).

If the filler is provided as an aqueous dispersion or sol, the amount of water in the aqueous dispersion or sol has to be taken into account when the amount of water and filler in the composition is calculated or determined.

Suitable non acid-reactive fillers are also commercially available as aqueous dispersions from e.g. Obermeier, Bad Berleburg, Germany under the trade name Levasil™, including type “50/50%”, wherein the % value indicates the filler content in wt. %.

If desired, the surface of the particles of the non acid-reactive fillers can be surface treated. Suitable surface-treating agents include silanes, e.g. trimethoxysilanes carrying an organic functional group to modify the chemical properties of the particles. Suitable silanes are e.g. silanes to modify the acidic properties (carrying amino groups or carrying carboxylic acid groups) or silanes to modify the hydrophobicity/hydrophilicity (carrying an alkane chain or carrying a polyethylene glycol chain).

Non acid-reactive fillers may also be x-ray visible, including particles of metal oxides and metal fluorides. Oxides or fluorides of heavy metals having an atomic number greater than 28 or 30 and less than 72 can be preferred. Suitable metal oxides are the oxides of yttrium, strontium, barium, zirconium, hafnium, niobium, tantalum, tungsten, bismuth, molybdenum, tin, zinc, lanthanide elements (i.e. elements having atomic numbers ranging from 57 to 71, inclusive), cerium and combinations thereof.

Particularly preferred radiopacifying metal oxides include lanthanum oxide, zirconium oxide, yttrium oxide, ytterbium oxide, barium oxide, strontium oxide, cerium oxide, and combinations thereof.

Suitable metal fluorides are e.g. yttrium trifluoride and ytterbium trifluoride.

According to one embodiment, the non acid-reactive filler is selected from silica, (alumo)silicates, alumina and mixtures thereof.

The non acid-reactive filler is typically present in the following amounts: at least 0 or 4 or 6 wt. %; utmost 40 or 35 or 30 wt. %; range: 0 to 40 or 4 to 35 or 6 to 30 wt. %; wt. % with respect of the weight of the dental composition.

The dental composition may also comprise a polyacid.

The polyacid should have a molecular weight sufficient to provide good storage, handling, and mixing properties, as well as to yield good material properties in the glass ionomer material.

According to one embodiment, the polyacid can be characterized by the following features alone or in combination: being a solid (at 23° C.); molecular weight (Mw): 2,000 to 250,000 or 4,000 to 100,000 g/mol (evaluated against a polyacrylic acid sodium salt standard using gel permeation chromatography).

If the molecular weight of the polyacid is too high, obtaining a workable consistency of the obtained paste when mixing the compositions contained in the kit of parts described in the present text might become difficult. Furthermore, preparation of the compositions might become difficult, too. In addition, the obtained mixture or composition might become too sticky (i.e. adheres to the dental instrument used for application).

If the molecular weight of the polyacid is too low, the viscosity of the obtained paste might become too low and the mechanical properties inferior.

Typically, the polyacid is a polymer having a plurality of acidic repeating units.

The polyacid to be used for the cement composition described in the present text is substantially free of polymerizable groups.

The polyacid need not be entirely water soluble, but typically it is at least sufficiently water-miscible so that it does not undergo substantial sedimentation when combined with other aqueous components.

The polyacid is hardenable in the presence of, for example, an acid-reactive glass and water, but does not contain ethylenically unsaturated groups.

That is, the polyacid is a polymer obtained by polymerising an unsaturated acid. However, due to the production process, a polyacid might still contain unavoidable traces of free monomers (e.g. up to 1 or 0.5 or 0.3 wt. % with respect to the amount of monomers used).

Typically, the unsaturated acid is an oxyacid (i.e., an oxygen containing acid) of carbon, sulfur, phosphorous, or boron. More typically, it is an oxyacid of carbon.

Suitable polyacids include, for example, polyalkenoic acids such as homopolymers and copolymers of unsaturated mono-, di-, or tricarboxylic acids.

Polyalkenoic acids can be prepared by the homopolymerization and copolymerization of unsaturated aliphatic carboxylic acids, e.g., acrylic acid, methacrylic acid, itaconic acid, maleic acid, glutaconic acid, aconitic acid, citraconic acid, mesaconic acid, fumaric acid, and tiglic acid.

Suitable polyacids also include alternating copolymers of maleic acid and ethylene (e.g. in a molar one to one ratio).

Suitable polyacids are also described in the following documents: U.S. Pat. No. 4,209,434 (Wilson et al.), U.S. Pat. No. 4,360,605 (Schmitt et al.). The content of these documents with respect to the description of the polyacid is herewith incorporated by reference.

Suitable polyacids are also included as aqueous solutions in the liquid component of commercially available products from e.g. 3M Oral Care (e.g. Ketac™ Fil Plus Handmix) or GC (e.g. Fuji™ IX GP Handmix).

The amount of polyacid should be sufficient to react with the acid-reactive glass and to provide an ionomer composition with desirable hardening properties.

If the amount of polyacid is too high, obtaining a workable consistency of the obtained paste when mixing the compositions contained in the kit of parts described in the present text might become difficult. Furthermore, preparation of the compositions might become difficult. In addition, the obtained mixture or composition might become too sticky (i.e. adheres to the dental instrument used for application).

The polyacid is typically present in the following amounts: at least 1 or 4 or 6 wt. %; utmost 25 or 20 or 15 wt. %; range: 1 to 25 or 4 to 20 or 6 to 15 wt. %; wt. % with respect of the weight of the dental composition.

The dental composition may also comprise water. Typically de-ionised water is used.

If the amount of the water is too low, the curing of the glass ionomer cement reaction might be affected.

If the amount of water is too high, obtaining a workable consistency of the obtained paste might become difficult. Furthermore, it might become difficult to achieve the desired mechanical properties and the paste might separate during storage.

Water is typically present in the following amounts: at least 0.25 or 0.5 or 1 wt. %; utmost 20 or 15 or 10 wt. %; range: 0.25 to 20 or 0.5 to 15 or 1 to 10 wt. %; wt. % with respect of the weight of the dental composition.

The dental composition can also comprise additive(s).

Additive(s) which can be added include dyes, pigments, photo-bleachable colorants, stabilizers plastizisers, retarders and mixtures thereof.

Examples of dyes or pigments, which can be used include titanium dioxide or zinc sulphide (lithopones), red iron oxide 3395, Bayferrox™ 920 Z Yellow, Neazopon™ Blue 807 (copper phthalocyanine-based dye) or Helio™ Fast Yellow ER. These additives may be used for individual colouring of the dental compositions.

Examples of photo-bleachable colorants which can be present include Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein and blends thereof. Further examples of photo-bleachable colorants can be found in U.S. Pat. No. 6,444,725.

Further additives, which can be added, include stabilizers, especially free radical scavengers such as substituted and/or unsubstituted hydroxyaromatics (e.g. butylated hydroxytoluene (BHT), hydroquinone, hydroquinone monomethyl ether (MEHQ), 3,5-di-tert-butyl-4-hydroxyanisole (2,6-di-tert-butyl-4-ethoxyphenol), 2,6-di-tert-butyl-4-(dimethylamino)methylphenol or 2,5-di-tert-butyl hydroquinone, 2-(2′-hydroxy-5′-methylphenyl)-2H-benzotriazole, 2-(2′-hydroxy-5′-t-octylphenyl)-2H-benzotriazole, 2-hydroxy-4-methoxybenzophenone (UV-9), 2-(2′-hydroxy-4′,6′-di-tert-pentylphenyl)-2H-benzotriazole, 2-hydroxy-4-n-octoxybenzophenone, 2-(2′-hydroxy-5′-methacryloxyethylphenyl)-2H-benzotriazole, and phenothiazine.

Further additives, which can be added, include retarder(s), (such as 1,2-diphenylethylene), and plasticizers (including polyethylene glycol derivatives, polypropylene glycols, low-molecular-weight polyesters, dibutyl, dioctyl, dinonyl and diphenyl phthalate, di(isononyl adipate), tricresyl phosphate, paraffin oils, glycerol triacetate, bisphenol A diacetate, ethoxylated bisphenol A diacetate, and silicone oils).

There is no need for additives to be present, so additives might not be present at all. However, if they are present, they are typically present in an amount which is not detrimental to the intended purpose.

Additives are typically present in the following amounts: at least 0 or 0.05 or 0.1 wt. %; utmost 15 or 10 or 5 wt. %; range: 0 to 15 or 0.05 to 10 or 0.1 to 5 wt. %; wt. % with respect of the weight of the dental composition.

In certain embodiments the dental composition comprises the respective components in the following amounts:
polymerizable components with acidic moiety: 0.5 to 15 wt. %,
polymerizable components without acidic moiety: 1 to 50 wt. %,
polyacid: 1 to 25 wt. %,
water: 0.25 to 20 wt. %,
acid-reactive glass: 5 to 70 wt. %,
non acid-reactive filler: 0 to 40 wt. %,
a Ca-ions releasing component: 0.5 to 30 wt. %,
a F-ions releasing component: 0.1 to 20 wt. %,
redox-initiating system comprising an oxidizing agent, a reducing agent and optionally a transition metal component: 0.1 to 5 wt. %,
photo-initiator: 0 to 2 wt. %,
additives: 0 to 15 wt. %,
wt. % with respect of the weight of the dental composition.

In certain embodiments the dental composition comprises the respective components in the following amounts:
polymerizable components with acidic moiety: 1 to 10 wt. %,
polymerizable components without acidic moiety: 5 to 40 wt. %,
polyacid: 4 to 20 wt. %,
water: 0.5 to 15 wt. %,
acid-reactive glass: 5 to 50 wt. %,
non acid-reactive filler: 4 to 35 wt. %,
a Ca-ions releasing component: 1 to 25 wt. %,
a F-ions releasing component: 0.5 to 15 wt. %,
redox-initiating system comprising an oxidizing agent, a reducing agent and optionally a transition metal component: 0.3 to 5 wt. %,
photo-initiator: 0.01 to 1 wt. %,
additives: 0 to 10 wt. %,
wt. % with respect of the weight of the dental composition.

The dental composition is typically provided as a kit of parts comprising an acid part or paste and a non-acidic part or paste.

The acid part and non-acidic part are typically provided in a ratio of 3:1 to 1:3 or 2:1 to 1:2 with respect to volume. A ratio of 1:1 with respect to volume can sometimes be preferred.

The components of the kit of parts are typically provided in a form avoiding an undesired reaction or interaction among them.

The acidic part or paste typically comprises
polymerizable components with acidic moiety, preferably in an amount of 1 to 20 wt. %,
optionally polymerizable components without acidic moiety, preferably in an amount of 0 to 50 or 5 to 40 wt. %,
hydroperoxide component, preferably in an amount of 0.1 to 5 wt. %,
optionally transition metal component, preferably in an amount of 0 to 0.5 or 0.001 to 0.5, polyacid, preferably in an amount of 1 to 30 wt. %,
optionally non acid-reactive filler, preferably in an amount of 0 to 50 or 5 to 50 wt. %,
water, preferably in an amount of 0.5 to 20 or 1 to 20 wt. %,
optionally additive(s) in an amount of 0 to 10 wt. %,
wt. % with respect to the amount of the acidic part or paste.

The non-acidic part or paste typically comprises
polymerizable components without acidic moiety, preferably in an amount of 1 to 60 wt. % or 5 to 40 wt. %,
optionally a photo-initiator, preferably in an amount of 0 to 2 wt. % or 0.01 to 1 wt. %
polymerizable thiourea component, preferably in an amount of 0.1 to 3 wt. %,
acid-reactive glass, preferably in an amount of 15 to 70 wt. %,
optionally non-acid-reactive filler, preferably in an amount of 0 to 30 or 1 to 20 wt. %,
optionally additive(s), preferably in an amount of 0 to 10 wt. %,
Ca-ions releasing component, preferably in an amount of 5 to 50 wt. %,
F-ions releasing component, preferably in an amount of 1 to 30 wt. %,
with respect to the non-acidic part or paste.

According to some embodiments, the dental composition does not comprise the following components alone or in combination:
NaF in an amount of more than 0.5 or 0.2 wt. %;
AlF₃ anhydrous in an amount of more than 0.5 or 0.2 wt. %;
CaCO₃ in an amount of more than 0.5 or 0.2 wt. %;
CaCl₂ in an amount of more than 0.5 or 0.2 wt. %;
Ca(OH)₂ in an amount of more than 0.5 or 0.2 wt. %;
   wt. % with respect to the weight of the dental composition.

The presence of these components may increase the release of calcium and fluoride ions from the dental composition in an undesired manner with the result that the solubility product of calcium fluoride is exceeded and insoluble calcium fluoride is formed.

The dental composition can be used by combining the respective components and mixing the composition. If desired, a speed-mixer or kneading machine can be used. As appropriate the production is done under save-light conditions.

The dental composition described in the present text is typically stored in an adequate packaging material or device.

If the dental composition is provided as a kit of parts comprising two different parts or pastes, the parts or pastes may be contained in separate sealable vessels or receptacles (e.g. made out of plastic or glass).

For use, the practitioner may take adequate portions of the compositions contained from the vessels and mix the portions by hand on a mixing plate.

According to a preferred embodiment, the different parts or pastes are contained in separate compartments of a storing device.

The storing device typically comprises two compartments for storing the respective parts, each compartment being equipped with a nozzle for delivering the respective part. Once delivered in adequate portions, the parts can then be mixed by hand on a mixing plate.

According to another preferred embodiment, the storing device has an interface for receiving a static mixing tip. The mixing tip is used for mixing the respective pastes. Static mixing tips are commercially available e.g. from SulzerMixpac company.

Suitable storing devices include cartridges, syringes and tubes.

The storing device typically comprises two housings or compartments having a front end with a nozzle and a rear end and at least one piston movable in the housing or compartment.

Cartridges which can be used are described e.g. in US 2007/0090079 A1 (Keller) or U.S. Pat. No. 5,918,772 (Keller et al.), the disclosure of which is incorporated by reference. Some of the cartridges which can be used are commercially available e.g. from Sulzer Mixpac AG (Switzerland). Static mixing tips which can be used are described e.g. in US 2006/0187752 A1 (Keller) or in U.S. Pat. No. 5,944,419 (Streiff), the disclosure of which is incorporated by reference. Mixing tips which can be used are commercially available from Sulzer Mixpac AG (Switzerland), as well.

Other suitable storing devices are described e.g. in WO 2010/123800 (3M), WO 2005/016783 (3M), WO 2007/104037 (3M), WO 2009/061884 (3M), in particular the device shown in FIG. 14 of WO 2009/061884 (3M) or WO 2015/073246 (3M), in particular the device shown in FIG. 1 of WO 2015/07346. Those storing devices have the shape of a syringe. The content of these references is herewith incorporated by reference, as well.

Alternatively, but less preferred, paste/paste compositions described in the present text can be provided in two individual syringes and the individual pastes can be mixed by hand prior to use.

Thus, the invention is also directed to a device for storing the kit of parts described in the present text, the device comprising two compartments, Compartment A and Compartment B, Compartment A containing the non-acidic part or paste and Compartment B containing the acidic part or paste, the non-acidic part or paste and the acidic part or paste being as described in the present text, Compartment A and Compartment B both comprising a nozzle or an interface for receiving an entrance orifice of a static mixing tip.

The mixing ratio of the non-acidic part or paste and the acidic part or paste is typically 3:1 to 1:3 with respect to volume, preferably 2:1 to 1:2, more preferably 1:1 with respect to volume.

The content of the above mentioned references is herewith incorporated by reference.

The invention also relates to a kit of parts.

The kit of parts comprises the dental composition described in the present text and the following items alone or in combination: dental milling block; dental crown; mixing tips; dental adhesive or bonding system; conditioning liquid for crown material or tooth material.

Suitable dental milling blocks typically comprise a porous zirconia material, which contains yttria as a phase-stabilizing component and colouring components. Examples of dental milling blocks are described in US2017/020639 (Jahns et al.), US2015/238291A1 (Hauptmann et al.).

Suitable dental adhesives are acidic dental composition with a rather low viscosity (e.g. 0.01 to 3 Pa*s at 23° C.). Dental adhesives directly interact with the enamel or dentin surface of a tooth. Dental adhesives are typically one-part compositions, are radiation-curable and comprise ethylenically unsaturated component(s) with acidic moiety, ethylenically unsaturated component(s) without acidic moiety, water, sensitizing agent(s), reducing agent(s) and additive(s). Examples of dental adhesives are described in US2020/0069532A1 (Thalacker et al.) and US2017/0065495A1 (Eckert et al.), US2019/231494A1 (Dittmann et al.).

Thus, the kit of parts contains parts or components which can be used together in a process for restoring a defect tooth.

The dental milling block is used for machining a dental restoration, the dental adhesive is used for treating the surface of tooth to be restored and the dental composition described in the present text is used for cementing the dental restoration machined from the dental mill block.

Prefabricated dental crowns which can be used include stainless steel crowns (3M Oral Care) or plastic crowns made out of polyacetal, polyacrylate, polymethacrylate (PMMA), polyaryletherketone (PAEK), polyetherketon (PEK), polyetheretherketon (PEEK), polyetherimide (PEI), polyethersulfone (PES) and polysulfone (PSU).

Suitable prefabricated dental crown are also described in U.S. Pat. No. 8,651,867B2 (Zilberman), WO2007/098485A2 (Nusmile), WO2008/033758A2 (3M), and US2007/0196792A1 (Johnson et al.). The content of the above mentioned references is herewith incorporated by reference.

Commercially available dental adhesives or bonding systems which can be used include Scotchbond™ Universal Adhesive (3M Oral Care), Scotchbond™ Universal Plus Adhesive (3M Oral Care).

Commercially available conditioners which can be used include Ketac™ Conditioner (3M Oral Care) and Cavity Conditioner (GC).

The dental composition is typically provided as RM-GIC.

The dental composition is typically used for cementing dental restorations to the surface of prepared tooth. The dental restoration typically has the shape of a dental crown or bridge, veneer, onlay or inlay. The material of the dental restoration may comprise, essentially consist of or consist of a ceramic (e.g. zirconia, alumina), glass-ceramic (e.g. lithium-disilicate), metal (e.g. gold), metal-alloy or a composite material.

Due to the presence of Ca-ions and F-ions releasing components, the dental composition may be useful in contributing to the remineralization process of a defect tooth in the mouth of a mammal by releasing calcium and fluoride ions to the surface of the defect tooth.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate the invention.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Viscosity of Single Parts

If desired, the viscosity of the single parts/pastes of the composition can be measured using a Physica MCR 301 Rheometer (Anton Paar, Graz, Austria) with a plate/plate geometry under controlled shear rate at 23° C. The plate diameter is 15 mm, the separation gap between the plates 0.5 mm. The shear rate is 20 s⁻¹.

### Viscosity of Two-part Composition

If desired, the viscosity of the two-part composition can be measured using a Physica MCR 301 Rheometer (Anton Paar, Graz, Austria) with a plate/plate geometry under controlled oscillation at 28° C. The plate diameter is 8 mm, the separation gap between the plates 0.75 mm. The oscillation frequency is 1.25 Hz and deflection is 1.75%.

### Particle Size (Suitable for Micro-Sized Particles)

If desired, the particle size distribution including the particle size (d50) per volume can be determined by laser diffraction with a Mastersizer 2000 (Malvern) particle size detection device applying the Fraunhofer approximation. During the measurement, ultrasonic is typically used to accurately disperse the sample. For water-insoluble particles, water is typically used as dispersant.

### Particle Size (Suitable for Nano-Sized Particles)

If desired, particle size measurements can made using a light scattering particle sizer equipped with a red laser having a 633 nm wavelength of light (obtained under the trade designation “ZETA SIZER—Nano Series, Model ZEN3600” from Malvern Instruments Inc., Westborough, MA). Each sample is analyzed in a one-centimeter square polystyrene sample cuvette. The sample is diluted 1:100, e.g. 1 g of sample is given to 100 g of de-ionized water and mixed. The sample cuvette is filled with about 1 gram of diluted sample. The sample cuvette is then placed in the instrument and equilibrated at 25° C. The instrument parameters are set as follows: dispersant refractive index 1.330, dispersant viscosity 0.8872 mPa*s, material refractive index 1.43, and material absorption value 0.00 units. The automatic size-measurement procedure is then run. The instrument automatically adjusts the laser-beam position and attenuator setting to obtain the best measurement of particle size.

The light scattering particle-sizer illuminates the sample with a laser and analyzes the intensity fluctuations of the light scattered from the particles at an angle of 173 degrees. The method of Photon Correlation Spectroscopy (PCS) can be used by the instrument to calculate the particle size. PCS uses the fluctuating light intensity to measure Brownian motion of the particles in the liquid. The particle size is then calculated to be the diameter of sphere that moves at the measured speed.

The intensity of the light scattered by the particle is proportional to the sixth power of the particle diameter. The Z-average size or cumulant mean is a mean calculated from the intensity distribution and the calculation is based on assumptions that the particles are mono-modal, mono-disperse, and spherical. Related functions calculated from the fluctuating light intensity are the Intensity Distribution and its mean. The mean of the Intensity Distribution is calculated based on the assumption that the particles are spherical. Both the Z-average size and the Intensity Distribution mean are more sensitive to larger particles than smaller ones.

The Volume Distribution gives the percentage of the total volume of particles corresponding to particles in a given size range. The volume-average size is the size of a particle that corresponds to the mean of the Volume Distribution. Since the volume of a particle is proportional to the third power of the diameter, this distribution is less sensitive to larger particles than the Z-average size. Thus, the volume-average will typically be a smaller value than the Z-average size. In the scope of this document the Z-average size is referred to as “mean particle size”.

### pH Value

If desired, the pH value of a paste of can be determined as follows: a wet pH sensitive paper or test stick is brought in contact with the composition to be analysed.

### Buffer Solution Test

0.4 g of the material to be tested was added to a glass beaker. The beaker was then charged with 24 mL of de-ionized water and 16 mL of pH 4 buffer solution (BDH5018, VWR International, Radnor, PA) with a 800 Dosino dosing unit (Metrohm, Herisau, Switzerland) and mechanically stirred using an 802 stirrer unit (Metrohm). The pH of the solution was continuously measured using an InLab Expert pH meter (51343100, Mettler Toledo, Columbus, OH) until the pH of the solution rose above 8, at which point the test was ended and the time to pH=8 was recorded.

### Shear Bond Strength (SBS)

Bovine teeth were ground flat to expose dentin, polished (grit 320 sandpaper), water-rinsed, and gently air-dried. Sandblasted and silanized steel rods (diameter=4 mm) were cemented onto the prepared teeth using a mixture of the pastes to be examined. A load of 20 g/mm² was put onto the cemented rods for 10 min at 36° C. and then removed. After that the specimens were stored for 22 h. Then, specimens were subjected to shear bond strength (SBS) testing using a Zwick 010 (Germany) tensile testing machine (speed=0.75 mm/min).

### Determination of Fluoride and Calcium Release of Individual Components

2.5 g Ca-ions releasing component is steered in 50 ml de-ionized water having a pH of 2 for 24 hours at a temperature of 23° C. The concentration of the Ca-ions is determined afterwards with a Ca-ions selective electrode.

2.5 g F-ions releasing component is steered in 50 ml de-ionized water having a pH of 7 for 24 hours at a temperature of 23° C. The concentration of the F-ions is determined afterwards with a Ca-ions selective electrode.

The F-ions and Ca-ions releasing capacity (in ppm; mg/l) of certain components is given in Table 2 below.

### Determination of Fluoride and Calcium Release of Cured Samples

The release of calcium ions was determined using an inductively coupled plasma optical spectrometer (ICP; Optima™ 8000, Perkin Elmer®). The release of fluoride was determined using a fluoride-selective electrode (FSE; Titrando™, Metrohm™).

ICP: First, three standard solutions were measured for calibration (eluent: 65% HNO₃). Then, testing of the extractant was performed. Two disc-like specimens (diameter 15 mm; height 1.5 mm) were prepared by hand-mixing (SF-S:SF-G=1.00:1.15 weight/weight) on a mixing pad for 20 s. The specimens were clamped and cured at 36° C., 100% humidity for 1 h. Then, the samples were demolded and stored for 23 h at 36° C. Afterwards, two disc-like specimens (total weight of about 1.4 g) were put into 50 ml of de-ionized water and stored at 36° C. After 1 day, the extractants were analyzed. The sample was atomized and ionized in argon plasma. The individual elements were excited by interaction with the excited state argon in the plasma. As each atom returned to its ground state from the excited state, they emitted light at wavelengths characteristic of the elements from which they originate. After testing, the two disc-like specimens were again immersed in fresh de-ionized water until next testing.

FSE: First, four standard (buffered) solutions were measured for calibration. The reference electrode was Ag/AgCl. Two disc-like specimens (diameter 15 mm; height 1.5 mm) were prepared by hand-mixing (SF-S:SF-G=1.00:1.15 weight/weight) on a mixing pad for 20 s. The specimens were clamped and cured at 36° C., 100% humidity for 1 h. Then, the samples were demolded and stored for 23 h at 36° C. Afterwards, two disc-like specimens (total weight of about 1.4 g) were put into 50 ml of de-ionized water and stored at 36° C. After 1 day, the extractants were analyzed. 10 ml of the sample solution were mixed with 10 ml of the buffer solution and testing was performed.

The amounts of ions were determined after 1 day, 1 week, and 1 month. Accumulated values after 1 month are given in Table 6.

### Water Contact Angel

If desired, the water contact angel can be determined as follows: a drop of a 10 wt. % ethanolic solution of the component to be tested is applied onto the surface of a dental mixing pad. The ethanolic solvent is evaporated to obtain a coated surface (approx. size: 4 cm²). On that surface a drop of water is placed and the development of the water-contact angle is analysed at 23° C. (Kruess Advance Software 1.13.1.31401). The average value obtained within 0 to 12 s after placement of the drop is taken.

### Setting Time

If desired, the setting time can be determined by recording the viscosity of the mixed composition during curing over a certain time period. Viscosity is measured using a Physica MCR 301 Rheometer (Anton Paar, Graz, Austria) with a plate/plate geometry at 28° C. From the recorded graph 2 measuring points are taken, which show the beginning and the end of curing. The two measuring points are:
A) Beginning of curing=Time when viscosity is three times the starting viscosity, which is determined 60 s after start of mixing.
B) Setting Time=Time when shear stress reaches 100,000 Pa.

TABLE 1
Materials

| Compound | | Description |
|---|---|---|
| Hydroxy ethyl methacrylate | HEMA | Monomer |
| Glycerine-1,3-dimethacrylate | GDMA | Monomer |
| Urethane dimethacrylate | UDMA | Monomer |
| Glycerol dimethacrylate phosphate | GDMP | Adhesive monomer |
| Water, de-ionized | Water | Water |
| Poly(acrylic acid-co-maleic acid) co-polymer | PAA | Polyacrylic acid |
| (1:1 co-polymer), Mw = 20,000 g/mol | | |
| 10% 2,6-di-tert-butyl 4-hydroxy toluene in HEMA | Stabsol 1 | Stabilizer |
| 10% 4-Hydroxy-2,2,6,6-tetramethylpiperidine 1-oxyl | Stabsol 2 | Stabilizer |
| (4-Hydroxy-TEMPO) in GDMA | | |
| 2.5% 4-Hydroxy-2,2,6,6-tetramethylpiperidine 1-oxyl | Stabsol 3 | Stabilizer |
| (4-Hydroxy-TEMPO) in GDMA | | |
| N-(5-methacryloxypentyl)-N′-ethyl thiourea | MOPETU | Reductant |
| Tetramethylbutyl hydroperoxide | TMBH-L | Oxidant |
| 1% copper procetonate in HEMA | Cu-catalyst | Catalyst |
| 1% Irgacure ™ 819 in GDMA | Photosol | Photo initiator |
| Aerosil ® R805 | R805 | Non acid-reactive filler |
| Aerosil ® OX50 | OX50 | Non acid-reactive filler |
| FAS glass 0.3% silanized, <63 μm (*) | FAS glass | Acid-reactive glass |
| YbF₃, 200 nm | YbF₃ | Radiopaque filler |
| Encapsulated CaO filler, 20 min BST (**) | CaO-Encap | Encapsulated Ca-ions |
| | | releasing component |

(*) acid-reactive FAS glass comparable to the commercially available glass G018-090 from Schott Glaswerke, Landshut, Germany;
(**) according to example 23 of WO 2018/102484A1.

TABLE 2

| F-Component | AlF₃ | AlF₃(1*H₂O) | AlF₃(3*H₂O) | NaF | KZnF₃ |
|---|---|---|---|---|---|
| F-ions release [ppm] | 11 | 1.280 | 2.740 | 19.880 | 2.180 |
| Ca-Component | CaF₂ | Ca(OH)₂ | CaSO₄(2*H₂O) | CaCl₂ | CaO-Encap |
| Ca-ions release [ppm] | 8 | 920 | 559 | 269.140 | 541 |
| F-Component | Na₃ AlF₆ | FAS glass | | | |
| F-ions release [ppm] | 219 | 70 | | | |
| Ca-Component | Ca₃(PO₄)₂ | | | | |
| Ca-ions release [ppm] | 326 | | | | |

### General Procedures

The pastes were prepared by first weighing in all the components and subsequent speed mixing.

### Example 1

The following pastes were prepared:

TABLE 3
Prepared non-acidic pastes

| | SF-G-287 | SF-G-288 | SF-G-292 |
|---|---|---|---|
| Compound | [wt. %] | [wt. %] | [wt. %] |
| UDMA | 3.4 | 3.4 | 3.4 |
| GDMA | 24.7 | 24.4 | 24.4 |
| Photosol | 3.0 | 3.0 | 3.0 |
| Stabsol 2 | 0.1 | 0.0 | 0.0 |
| Stabsol 3 | 0.0 | 0.4 | 0.4 |
| MOPETU | 1.1 | 1.1 | 1.1 |
| R805 | 6.0 | 6.0 | 6.0 |
| NaF | 0.0 | 0.0 | 7.8 |
| KZnF₃ | 0.0 | 10.0 | 0.0 |
| FAS glass | 46.8 | 36.8 | 39.0 |
| CaO-Encap | 15.0 | 15.0 | 15.0 |

(7.8 wt. % NaF provide the same amount of fluoride ions as 10 wt. % of KZNF₃, allowing for a fair comparison.)

TABLE 4
Prepared aqueous, acidic paste

| | SF-S-258 |
|---|---|
| Compound | Amount wt. % |
| HEMA | 18.8 |
| Water | 14.0 |
| GDMP | 9.6 |
| PAZ | 19.9 |
| TBMH-L | 1.2 |
| Stabsol 1 | 0.9 |
| Cu-catalyst | 0.6 |
| R805 | 10.6 |
| YbF₃ | 24.6 |

TABLE 5

| | | | | Mixing Ratio |
|---|---|---|---|---|
| | | Organic | Aqueous, | Organic |
| | Formulation | Paste | Paste | Paste (wt.):Aqueous |
| Example | (No.) | (No.) | (No.) | Paste (wt.) |
| 1 (CE) | SF-M-435 | SF-G-287 | SF-S-258 | 1.15:1.00 |
| 2 (CE) | SF-M-439 | SF-G-292 | SF-S-258 | 1.15:1.00 |
| 3 (IE) | SF-M-428 | SF-G-288 | SF-S-258 | 1.15:1.00 |

Prepared formulations;
CE: comparative example;
IE: inventive example

TABLE 6

| | | | | | F-Release | Ca-Release |
|---|---|---|---|---|---|---|
| | | | | CaO- | after 1 | after 1 |
| Exam- | NaF | KZnF₃ | F-Amount | Encap | month | month |
| ple | [wt. %] | [wt. %] | [wt. %] | wt. %] | [ppm] | [ppm] |
| 1 | 0.0 | 0.0 | 0.0 | 9.0 | 5.1 | 6.4 |
| 2 | 4.2 | 0.0 | 1.9 | 9.0 | 46.5 | 0.1 |
| 3 | 0.0 | 5.3 | 1.9 | 9.0 | 26.8 | 6.9 |

(4.2 wt. % NaF provide the same amount of fluoride ions as 5.3 wt. % of KZNF₃, allowing for a fair comparison.)

Using a combination of a complex F-ions releasing component and an encapsulated CaO containing filler provided a higher release of calcium ions than a combination of NaF and encapsulated CaO containing filler although the same amount of fluoride ions was added to both formulations (Example 2 and Example 3).

Without wishing to be bound by a certain theory it is assumed that the water solubility of the respective components may have an impact. The water solubility and/or ions-releasing capacity in particular of the F-ions releasing component should not be too high to reduce the risk of formation of insoluble CaF₂.

1. A dental composition comprising polymerizable components,
initiator suitable for curing the polymerizable components,
a Ca-ions releasing component having a Ca-ions releasing capability of 400 to 700 mg/l if 2.5 g Ca-ions releasing component is steered in 50 ml de-ionized water having a pH of 2 for 24 hours,
a F-ions releasing component having a F-ions releasing capability of 1,500 to 3,500 mg/l if 2.5 g F-ions releasing component is steered in 50 ml de-ionized water having a pH of 7 for 24 hours,
the amount of Ca-ions releasing component contained in the dental composition being higher than the amount of F-ions releasing component with respect to weight.

2. The dental composition according to claim 1, the Ca-ions releasing component comprising a basic core material that releases Ca-ions, and an inorganic shell material comprising a metal oxide surrounding the core.

3. The dental composition according to claim 1, the Ca-ions releasing component comprising a basic core material comprising a CaO component, in particular a calcium silicate such as 3CaO—SiO₂ and 2CaO—SiO₂, and an inorganic shell material comprising Al₂O₃ or SiO₂ surrounding the core.

4. The dental composition according to claim 1, the F-ions releasing component being characterized by the following features alone or in combination:
molecular weight: 110 to 300 g/mol; water solubility: 3 to 20 g/l water at 23° C.

5. The dental composition according to claim 1, the F-ions releasing component having the formula AₙMFₘ with
A being an alkali metal ion or NR₄⁺, with R being C₁ to C₁₈ alkyl, phenyl or substituted phenyl, M being selected from Ti, Zr, Al, Zn, P, n being 1 to 3, m being 3 to 6.

6. The dental composition according to claim 1, the F-ions releasing component being selected from KZnF₃, K₂TiF₆, and mixtures thereof.

7. The dental composition according to claim 1, the ratio of Ca-ions releasing component contained in the dental composition to F-ions releasing component being in a range of 1:0.9 to 1:0.1 with respect to weight.

8. The dental composition according to claim 1, the Ca-ions releasing component and the F-ions releasing component being present in the dental composition in amounts sufficient to enable a release of
F-ions in an amount of at least 10 ppm per 1 g of cured composition stored in de-ionized water over 30 days at 36° C., and
Ca-ions in an amount of at least 2 ppm per 1 g of cured composition stored in de-ionized water over 30 days at 36° C.

9. The dental composition according to claim 1, the initiator comprising a redox-initiator system comprising an oxidizing agent, a reducing agent, and optionally a transition metal component, the reducing agent being a thiourea component comprising a (meth)acrylate moiety.

10. The dental composition according to claim 1, the dental composition being a resin-modified glass ionomer cement composition comprising
polymerizable components with acidic moiety,
polymerizable components without acidic moiety,
polyacid,
water,
acid-reactive glass,
a Ca-ions releasing component comprising a basic core comprising a material that releases Ca-ions, and an inorganic shell material comprising a metal oxide surrounding the core,
a F-ions releasing component having the formula AₙMFₘ with A being an alkali metal ion or NR₄⁺, R being C₁ to C₁₈ alkyl, phenyl or substituted phenyl, M being selected from Ti, Zr, Al, Zn, P, n being 1 to 3, m being 3 to 6,
a redox-initiating system comprising an oxidizing agent, a reducing agent, and optionally a transition metal component,
optionally non acid-reactive filler,
optionally additive(s).

11. The dental composition according to claim 1, comprising the components in the following amounts:
polymerizable components with acidic moiety in an amount of 0.5 to 15 wt. %,
polymerizable components without acidic moiety in an amount of 1 to 50 wt. %,
polyacid in an amount of 1 to 25 wt. %,
acid-reactive glass in an amount of 5 to 70 wt. %,
water in an amount of 0.25 to 20 wt. %,
non acid-reactive filler in an amount of 0 to 40 wt. %,
Ca-ions releasing component comprising a basic core comprising a material that releases Ca-ions, and an inorganic shell material comprising a metal oxide surrounding the core, in an amount of 0.5 to 30 wt. %,
F-ions releasing component having the formula AₙMFₘ with A being an alkali metal ion or NR₄⁺, R being C₁ to C₁₈ alkyl, phenyl or substituted phenyl, M being selected from Ti, Zr, Al, Zn, P, n being 1 to 3, m being 3 to 6, in an amount of 0.1 to 20 wt. %,
redox-initiating system comprising an oxidizing agent, a reducing agent, and optionally a transition metal component, in an amount of 0.2 to 6 wt. %,
photo-initiator in an amount of 0 to 2 wt. %,
additives in an amount of 0 to 15 wt. %,
wt. % with respect to the weight of the whole composition.

12. The dental composition according to claim 1, not comprising the following components alone or in combination:
NaF in an amount of more than 0.5 wt. %;
AlF₃ anhydrous in an amount of more than 0.5 wt. %;
CaCO₃ in an amount of more than 0.5 wt. %;
CaCl₂) in an amount of more than 0.5 wt. %;
Ca(OH)₂ in an amount of more than 0.5 wt. %;
wt. % with respect to the whole composition.

13. The dental composition according to claim 1, being provided as a kit of parts comprising an acidic part and a non-acidic part, the acidic part and the non-acidic part being preferably provided in a ratio of 3:1 to 1:3 with respect to volume,
the acidic part comprising
polymerizable components with acidic moiety,
polymerizable components without acidic moiety,
an oxidizing component,
polyacid,
non acid-reactive filler,
water,
the non-acidic part comprising
polymerizable components without acidic moiety,
optionally a photo-initiator,
reducing agent, preferably a (polymerizable) thiourea reducing agent,
acid-reactive glass,
Ca-ions releasing component,
F-ions releasing component.

14. A kit of parts comprising the dental composition according to claim 1, including the following items alone or in combination: dental adhesive, dental milling block, prefabricated dental crown.

15. (canceled)

16. A kit of parts comprising the dental composition according to claim 13, including the following items alone or in combination: dental adhesive, dental milling block, prefabricated dental crown.
